(19) United States
(12) Patent Application Publication
(10) Pub. No.: US 2022/0175592 A1
(43) Pub. Date: Jun. 9, 2022

(54) AUXILIARY BAND AND METHOD FOR SECURING AN ABSORBENT PAD ONTO A VULVA REGION

(71) Applicant: Sandra Pantoja, Fairfield, CT (US)

(72) Inventor: Sandra Pantoja, Fairfield, CT (US)

(21) Appl. No.: 17/302,596

(22) Filed: May 7, 2021

Related U.S. Application Data
(60) Provisional application No. 63/122,944, filed on Dec. 9, 2020.

(51) Int. Cl. A61F 13/58; A61F 13/84; A61F 13/47

(52) U.S. Cl. CPC A61F 13/58; A61F 2013/4708; A61F 13/47; A61F 13/84

(57) ABSTRACT

An auxiliary band and method for securing an absorbent pad onto a vulva region of a female includes an elongated body, a first adhesive, and a second adhesive. The elongated body includes an inner surface adapted to face the vulva region. The inner surface defines a pad section adapted to receive the absorbent pad thereon and a peripheral portion extending along an outer periphery of the inner surface. The first adhesive is disposed on the pad section and is adapted to removably secure the absorbent pad to the pad section. The second adhesive is disposed at least on the peripheral portion and is adapted to removably attach the auxiliary band to the skin surrounding the vulva region.

### BACKGROUND OF THE INVENTION

Absorbent pads, such as menstrual pads, panty liners, incontinence absorbent articles, and the like, are commonly used to absorb bodily fluids, such as menstrual fluids and other vaginal discharges. The absorbent pads are designed to be worn inside an undergarment to absorb the bodily fluid, as it is expelled from the body of the wearer. While the absorbent pads are easy to use, they have certain limitations. For instance, the absorbent pads are not suitable for wearing during sports and/or underwater activities, such as swimming. Usually during such activities, the absorbent pads tend to shift and move resulting in leakage of the bodily fluid. Moreover, during swimming or any other underwater activity, the absorbent pads may absorb a lot of water and become bulky.

Other sanitary protection devices, such as tampons, may be used as an alternative during sports and/or underwater activities. Tampons are designed to be positioned internally within the vagina to collect bodily fluids. However, the process of insertion and removal of tampons inside the vagina may be uncomfortable; and physically and psychologically challenging.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 illustrates a top exploded view of an exemplary auxiliary band in accordance with some embodiments.

FIG. 2 illustrates an exemplary auxiliary band secured to a vulva region of a wearer, in accordance with some embodiments.

FIG. 3 is a front view of the auxiliary band secured to the vulva region of the wearer, in accordance with some embodiments.

FIG. 4 is a rear view of the auxiliary band secured to the vulva region of the wearer, in accordance with some embodiments.

FIG. 5 illustrates the exemplary auxiliary band worn inside a clothing of the wearer, in accordance with some embodiments.

FIG. 6 is an exploded view of an auxiliary band, in accordance with a second exemplary embodiment.

FIG. 7 is an exploded view of an auxiliary band, in accordance with a third exemplary embodiment.

FIG. 8 is an exploded view of an auxiliary band, in accordance with a fourth exemplary embodiment.

FIG. 9 illustrates an exemplary method for securing the auxiliary band onto the vulva region of the wearer, in accordance with some embodiments.

FIGS. 10A through 10F illustrate perspective views of the exemplary method for securing the auxiliary band onto the vulva region of the wearer, in accordance with some embodiments.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

In one aspect, an auxiliary band for securing an absorbent pad onto a vulva region of a female includes an elongated body, a first adhesive, and a second adhesive. The elongated body includes an inner surface adapted to face the vulva region. The inner surface defines a pad section adapted to receive the absorbent pad thereon and a peripheral portion extending along an outer periphery of the inner surface. The first adhesive is disposed on the pad section and is adapted to removably secure the absorbent pad to the pad section. The second adhesive is disposed at least on the peripheral portion and is adapted to removably attach the auxiliary band to the skin surrounding the vulva region.

In another aspect, a method for securing an absorbent pad onto a vulva region of a female includes providing an auxiliary band including an elongated body, a first adhesive, a second adhesive, and a protective cover. The elongated body has an inner surface for facing the vulva region. The inner surface defines a pad section for receiving the absorbent pad thereon and a peripheral portion extending along an outer periphery of the inner surface. The first adhesive is disposed on the pad section and is adapted to removably secure the absorbent pad to the pad section. The second adhesive is disposed at least on the peripheral portion and is adapted to removably attach the auxiliary band to the skin surrounding the vulva region. The protective cover covers the first adhesive and the second adhesive. The method further comprises peeling the protective cover to expose the first adhesive at the pad section and the second adhesive at the peripheral portion and attaching the absorbent pad to the exposed first adhesive. The method further comprises attaching the exposed second adhesive onto the skin surrounding the vulva region.

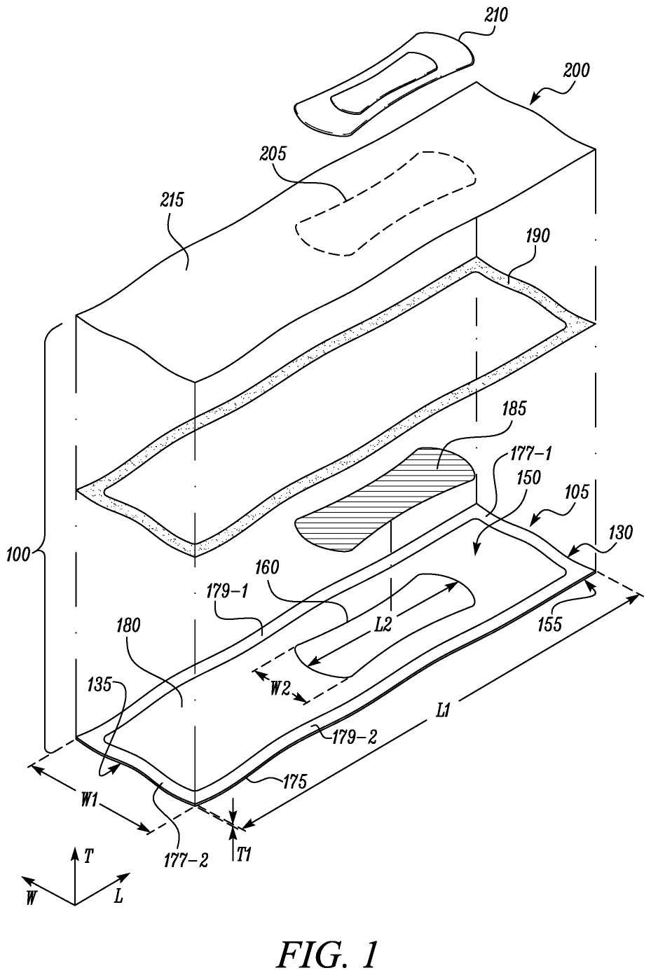

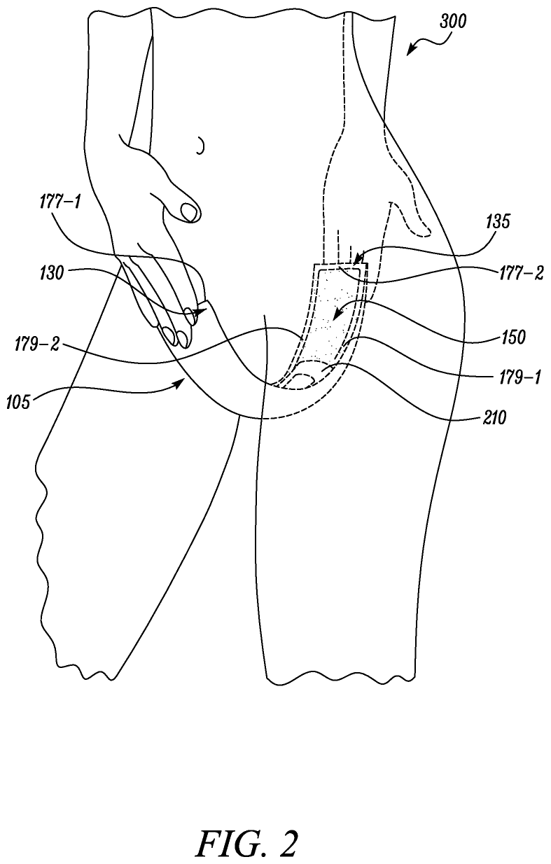

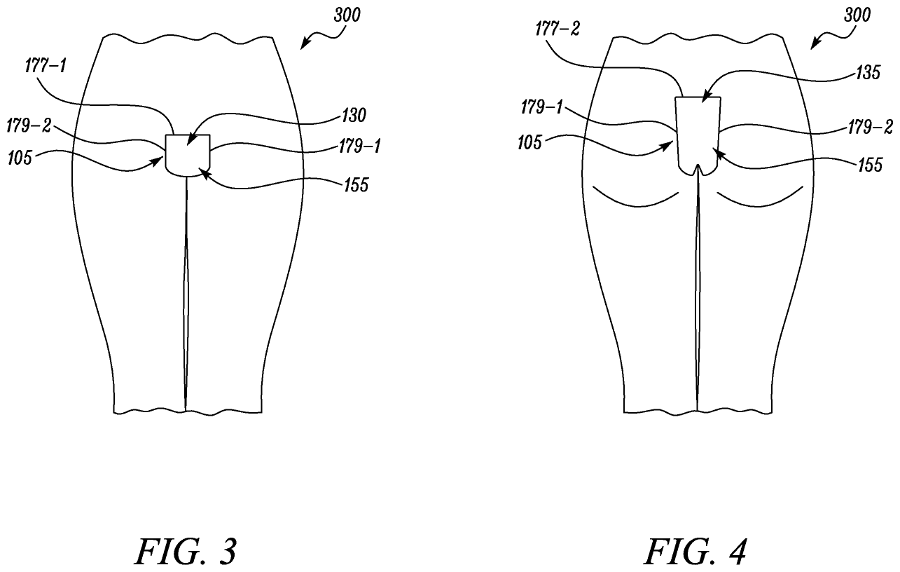

FIG. 1 illustrates a top exploded view of an exemplary auxiliary band 100, in accordance with various embodiments. The auxiliary band 100 may be configured to secure an absorbent pad, such as an absorbent pad 210, onto a vulva region of a female wearer, such as a wearer 300 (as shown in FIGS. 2 through 5). It will be appreciated by those of ordinary skill in the art that a female in some embodiments includes any living being having reproductive organs capable of bearing offspring or producing eggs. In some embodiments, a female further may be capable of ovulation and menstruation. For example, the absorbent pad 210 may include any absorbent article that is configured to adhere to the vulva region to absorb bodily fluids, such as menstrual fluids, urine, blood, and other vaginal discharges. It may be understood that “the vulva region”, also includes adjacent regions of the body of the female including the mons pubis region, pubic region, the perineal region, the tailbone region, and a surrounding portion of upper inner thighs.

In an embodiment, the auxiliary band 100 includes an elongated body 105 defining a longitudinal axis L, a lateral axis W, and vertical axis T. In one example, the auxiliary band 100 is rectangular in shape. Alternatively, the auxiliary band 100 may include any other elongated shape, such as oval, hourglass, and the like. The elongated body 105 of the auxiliary band 100 may be made from a waterproof material such as silicone, rubber, plastic, polyethylene, thermoplastic polyurethane, or any other waterproof material, or a combination thereof. In some other embodiments, the auxiliary band 100 may be made from any other material, such as medical grade body tape adhesives to achieve similar results without deviating from the scope of the claimed subject matter. Further, in some embodiments, the auxiliary band 100 can be made from a transparent and/or flexible material to enhance the aesthetic appeal.

In accordance with some embodiments, the elongated body 105 defines a length L1 along the longitudinal axis L, a width W1 along the lateral axis W, a thickness T1 along the vertical axis T. The length L1 and the width W1 of the auxiliary band 100 may be defined to cover the vulva region of the wearer 300, such as, based on the individual measurement requirements of the wearer 300. For example, the length L1 of the auxiliary band 100 may be greater than or equal to the individual measurements of the wearer 300 extending from a mons pubis region to a tailbone region. Similarly, the width W1 of the auxiliary band 100 may be greater than or equal to the measurement of separation between upper inner thighs adjacent to the vulva region of the wearer 300.

The elongated body 105 includes two spatially apart ends as illustrated as a front end 130 and a rear end 135 defined along the longitudinal axis L. For example, the front end 130 is configured to face and be secured to the mons pubis region during use while the rear end 135 is adapted to face and be secured to the tailbone region of the wearer 300 during use (as shown in FIGS. 2 through 4). The terms ‘front’ and ‘rear’, as used herein, are in relation to a direction of wearing of the auxiliary band 100, with said direction of wearing being exemplarily defined from the front end 130 towards the rear end 135 thereby securing the auxiliary band 100 from the mons pubis region towards the tailbone region of the wearer 300.

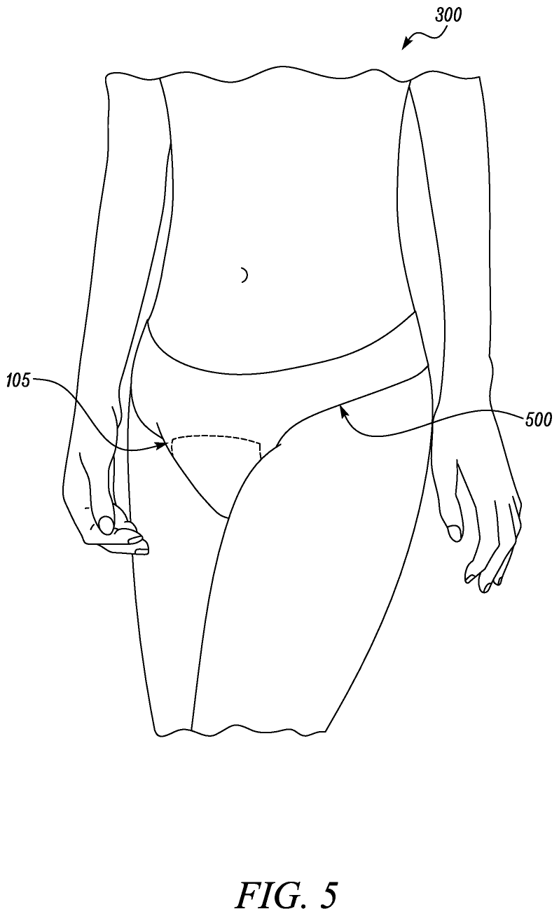

The elongated body 105 includes an inner surface 150 configured to face the vulva region of the wearer 300 and an outer surface 155 formed opposite to the inner surface 150 along the vertical axis T to define the thickness T1 of the auxiliary band 100. The outer surface 155 is configured to face outwards and away from the vulva region of the wearer 300. For example, the outer surface 155 is generally adapted to face towards or placed adjacent to an inner surface or an inner lining of a clothing, such as an undergarment 500 (as shown in FIG. 5), worn by the wearer 300.

In an exemplary embodiment, the inner surface 150 defines a pad section 160, a peripheral portion 175, and an intermediary portion 180 extending between the pad section 160 and the peripheral portion 175. The pad section 160 may be an elongated shaped section, that extends along the longitudinal axis L and is configured to receive the absorbent pad 210 thereon. In some embodiments, the pad section 160 is defined by adding visible indicators, such as, in the form of outline or colored section, on the inner surface 150 of the auxiliary band 100. The visible indicators may be configured to provide placement aid for suitable placement of the absorbent pad 210 over the pad section 160.

As shown, the pad section 160 defines a pad length L2 and a pad width W2, which may be varied based on the dimensions of the absorbent pad 210 desired to be worn by the wearer 300. In an exemplary embodiment, the pad length L2 is less than the length L1 of the elongated body 105, whereas the pad width W2 is less than or equal to the width W1 of the elongated body 105. In the illustrated example, the pad section 160 is positioned to extend towards and proximal to the front end 130. It will be appreciated that the position of the pad section 160 is merely exemplary and may be varied to achieve similar results.

The peripheral portion 175 extends along an outer periphery of the inner surface 150 and is adapted to be secured to the skin surrounding the vulva region of the wearer 300. The outer periphery may be defined to include the longitudinal edges 179-1, 179-2 of the inner surface 150 extending along the longitudinal axis L and lateral edges 177-1, 177-2 extending along the lateral axis W. Similarly, the intermediary portion 180 may be adapted to be in close contact with the skin of the wearer 300 in the vulva region.

The auxiliary band 100 further includes one or more types of adhesives for securing the absorbent pad 210 onto the pad section 160 and for securing the auxiliary band 100 onto the vulva region of the wearer 300. For example, as shown in FIG. 1, the auxiliary band 100 includes at least a first adhesive 185 disposed on the pad section 160 and a second adhesive 190 disposed at least on the peripheral portion 175. The first adhesive 185 may be adapted to removably secure the absorbent pad 210 onto the pad section 160, whereas the second adhesive 190 may be adapted to removably attach the inner surface 150 of the auxiliary band 100 to the skin surrounding the vulva region of the wearer 300.

In an exemplary embodiment, the second adhesive 190 disposed on the peripheral portion 175 is adapted to removably attach the front end 130, including, but not limited to the lateral edge 177-1 of the inner surface 150 to the mons pubis region, the rear end 135, including but not limited to the lateral edge 177-2 of the inner surface 150 to the tailbone region, and the longitudinal edges 179-1, 179-2 to the skin between the mons pubis region and the tailbone region of the wearer 300, such as the skin on the inner thighs (as shown in FIGS. 2 through 4). The second adhesive 190 along the peripheral portion 175 facilitates securing and sealing the auxiliary band 100 to the vulva region from all the sides.

In one embodiment (shown in FIG. 1), the first adhesive 185 and the second adhesive 190 are composed of different adhesive materials. For example, the first adhesive 185 may be made from any adhesive material capable of providing removable adhesion to the absorbent pad 210, whereas since the second adhesive 190 is adapted to attach to the skin of the wearer 300, it is made from body adhesive materials. Examples of the adhesive used for the first adhesive 185 may include, but are not limited to, silicone, kinesiology tape, polyethylene, thermoplastic polyurethane, hydrogels, hydrocolloids, acrylics based adhesives, or any other adhesive known or used in the art. Whereas examples of the body adhesive materials for the second adhesive 190 may include, but are not limited to, silicone, kinesiology tape, polyethylene, thermoplastic polyurethane, polymeric adhesive materials or any adhesive materials that can be used on the skin of the wearer 300 without causing any irritation or harm to the skin of the wearer 300. Further, in accordance with some embodiments, the first adhesive 185 and the second adhesive 190 are disposed separately on the pad section 160 and the peripheral portion 175, respectively, of the inner surface 150 of the auxiliary band 100, as discussed above. Alternatively, in some embodiments, the first adhesive 185 and the second adhesive 190 are formed directly on the pad section 160 and the peripheral portion 175, respectively, of the inner surface 150 of the auxiliary band 100.

In one exemplary embodiment, the first adhesive 185 and the second adhesive 190 may include reusable adhesives that are adapted to provide re-attachable adhesion for repeated use of the auxiliary band 100. For instance, the reusable first adhesive 185 disposed on the pad section 160 is adapted to provide re-attachable adhesion for removably securing the absorbent pad 210 onto the pad section 160. Similarly, the reusable second adhesive 190 is adapted to provide re-attachable adhesion for removably securing the peripheral portion 175 of the auxiliary band 100 to the skin surrounding the vulva region of the wearer 300.

The re-attachable adhesion obtained by using the reusable first adhesive 185 and second adhesive 190 allows for repeated application and use of the auxiliary band 100, without having to replace the auxiliary band 100 after every use. The re-attachable adhesion provided by the reusable first adhesive 185 on the pad section 160 allows the wearer 300 to repeatedly use the same auxiliary band 100 by replacing the used absorbent pad 210 with a fresh absorbent pad 210 on the pad section 160, as and when required. Similarly, the re-attachable adhesion provided by the reusable second adhesive 190 on the peripheral portion 175 allows the wearer 300 to attach the same auxiliary band 100 multiple times on their skin. In some cases, when the wearer 300 is not satisfied with the positioning of the absorbent pad 210 or the auxiliary band 100, the re-attachable adhesion also allows the wearer 300 to remove and reapply the absorbent pad 210 or the auxiliary band 100 for better and comfortable wearing.

Further, in some embodiments, the auxiliary band 100 also includes a protective cover 200 adapted to cover the inner surface 150 and the first adhesive 185 and the second adhesive 190 disposed thereon, when the auxiliary band 100 is not in use. The protective cover 200 facilitates retention of the adhesion properties of the first adhesive 185 and the second adhesive 190 and also protects the auxiliary band 100 and the first adhesive 185 and the second adhesive 190 from dirt or debris. In accordance with some embodiments, the protective cover 200 is a reusable cover that is adapted for repeated application and removal on the inner surface 150. In an exemplary embodiment, the protective cover 200 is composed of a releasable paper or alternatively a plastic sheet.

In accordance with some embodiments, the protective cover 200 includes a first cover portion 205 and a second cover portion 215. For example, the first cover portion 205 may be adapted to cover the first adhesive 185 disposed on the pad section 160 and the second cover portion 215 may be adapted to cover the second adhesive 190 disposed on the peripheral portion 175. As shown, the first cover portion 205 may be marked by scored lines on the protective cover 200 so as to facilitate peeling of the first cover portion 205 for use. Further, the second cover portion 215 is illustrated to cover the peripheral portion 175 as well as the intermediary portion 180. Alternatively, the second cover portion 215 may be implemented to cover only the peripheral portion 175 and the second adhesive 190 disposed thereon. Additionally, although the protective cover 200 is shown and described to include two separate cover portions, a person skilled in the art will appreciate that a single piece protective cover to cover the entire inner surface 150 may also be implemented within the scope of the claimed subject matter. Moreover, other designs of the protective cover may also be implemented to achieve similar results within the scope of the claimed subject matter.

When the auxiliary band 100 is to be used, the protective cover 200 may be removed to expose the first adhesive 185 and the second adhesive 190 for securing the absorbent pad 210 on the pad section 160 and attaching the auxiliary band 100 to the skin surrounding the vulva region of the wearer 300, respectively. In the illustrated example, the first cover portion 205 of the protective cover 200 is removed to expose the first adhesive 185 disposed on the pad section 160 and the second cover portion 215 is removed to expose the second adhesive 190 disposed on the peripheral portion 175.

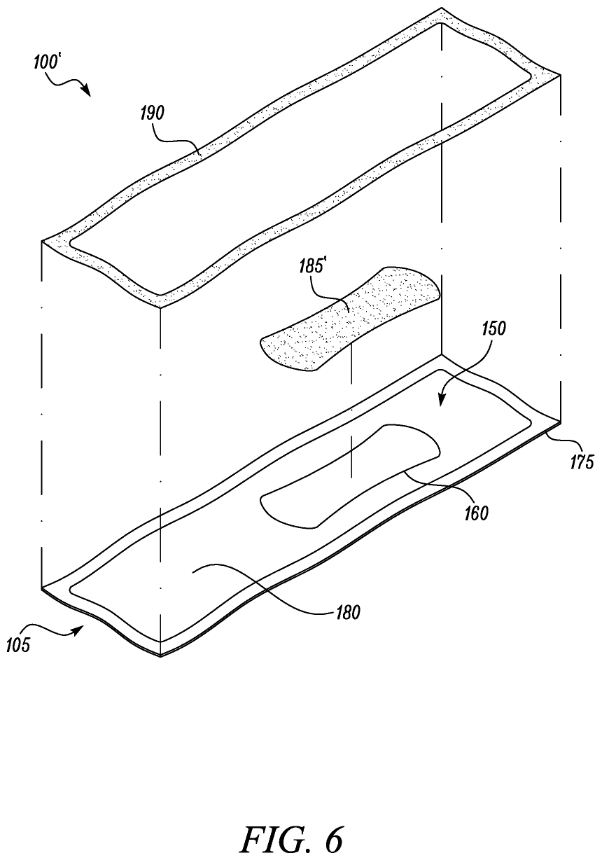

Referring to FIGS. 6 through 8, alternative embodiments of the auxiliary band 100 will now be described. FIG. 6 illustrates an exploded view of an exemplary auxiliary band 100′, according to a second embodiment. The auxiliary band 100′ is similar to the structure of the auxiliary band 100 with the exception that the first adhesive 185′ and the second adhesive 190 in auxiliary band 100′ are made from the same adhesive materials. In such an embodiment, both the first adhesive 185′ as well as the second adhesive 190 are made from body adhesive materials that can be used on the skin of the wearer 300 without causing any irritation or harm to the skin of the wearer 300.

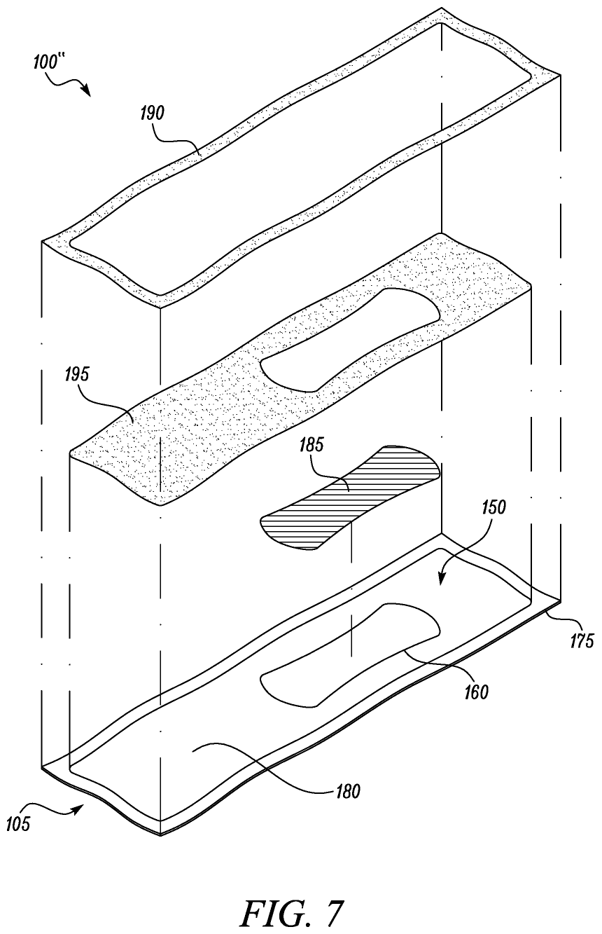

FIG. 7 illustrates an exploded view of an auxiliary band 100″, according to a third embodiment. In this embodiment, the auxiliary band 100″ includes a third adhesive 195 disposed on the intermediary portion 180 of the inner surface 150. The third adhesive 195 is adapted to attach the intermediary portion 180 to the skin surrounding the vulva region and provides extra adhesion for securing the inner surface 150 of the auxiliary band 100″ onto the skin of the wearer 300. As illustrated, the third adhesive 195 disposed on the intermediary portion 180 is made from the same material as the second adhesive 190 disposed on the peripheral portion 175. The third adhesive 195 also includes reusable adhesive that is adapted to provide re-attachable adhesion for repeated use of the auxiliary band 100″. Further, in accordance with some embodiments, the third adhesive 195 is also disposed separately on the intermediary portion 180 of the inner surface 150 of the auxiliary band 100. Alternatively, in some embodiments, the third adhesive 195 is formed directly on the intermediary portion 180 of the inner surface 150 of the auxiliary band 100.

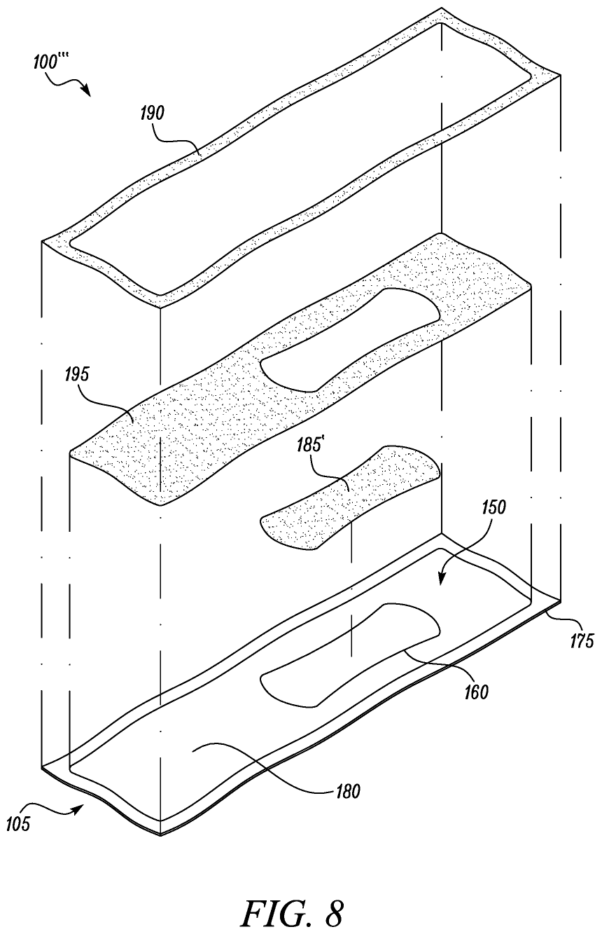

FIG. 8 illustrates an exploded view of an exemplary auxiliary band 100′″, according to a fourth embodiment. In this embodiment, the auxiliary band 100′″ includes the first adhesive 185′, the second adhesive 190, and a third adhesive 195, all made from the same adhesive material. In this case as well, each of the first adhesive 185′, the second adhesive 190, and the third adhesive 195 are made from reusable body adhesive materials.

In some alternative embodiments (not shown), one or more of the first adhesive 185, 185′, the second adhesive 190, and the third adhesive 195 may include a plurality of layers of respective adhesive materials. For such arrangements, each layer of the plurality of layers of adhesive material is adapted to be removed, after use, to expose another layer of adhesive lying underneath. For instance, when a top layer of adhesive material loses its adhesiveness or when the wearer 300 wishes to use a fresh layer of adhesive, another layer of adhesive lying underneath the top layer may be exposed by peeling off the top layer. With the other layer of adhesive exposed, the absorbent pad 210 may again be removably secured on the pad section 160 of the inner surface 150 and the peripheral portion 175 of the inner surface 150 may again be removably attached to the skin surrounding the vulva region of the wearer 300, as described above for subsequent use. The arrangement allows for the auxiliary band 100 and/or the absorbent pad 210 to be secured, removed and re-secured multiple times depending upon the number of the plurality of layers of adhesive material included in the auxiliary band 100.

In yet other alternative embodiments, one or more of the first adhesive 185, 185′, the second adhesive 190, and the third adhesive 195 may be provided as separate detachable adhesive bands that may be removably attached to the inner surface 150 of the elongated body 105 of the auxiliary band 100. In such arrangements, when the adhesiveness of any of the first adhesive 185, 185′, the second adhesive 190, and the third adhesive 195 is reduced after repetitive usage, they may be peeled off and replaced with a fresh adhesive band. For instance, the fresh adhesive band may include double-sided adhesives that are covered with peelable films on both sides. After peeling the film on the first side of the fresh adhesive band, the first side of the fresh adhesive may be secured to the inner surface 150 of the auxiliary band 100. After the first side of the fresh adhesive band is secured to the inner surface 150, the second side of the fresh adhesive may be peeled off to re-secure the absorbent pad 210 on the pad section 160 and/or the auxiliary band 100 to the skin of the wearer 300, in a similar manner as explained above.

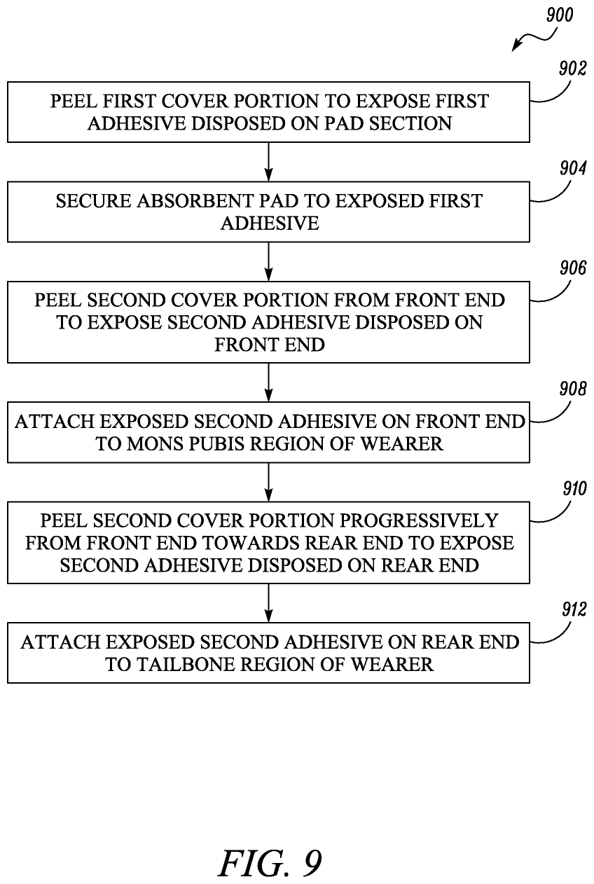

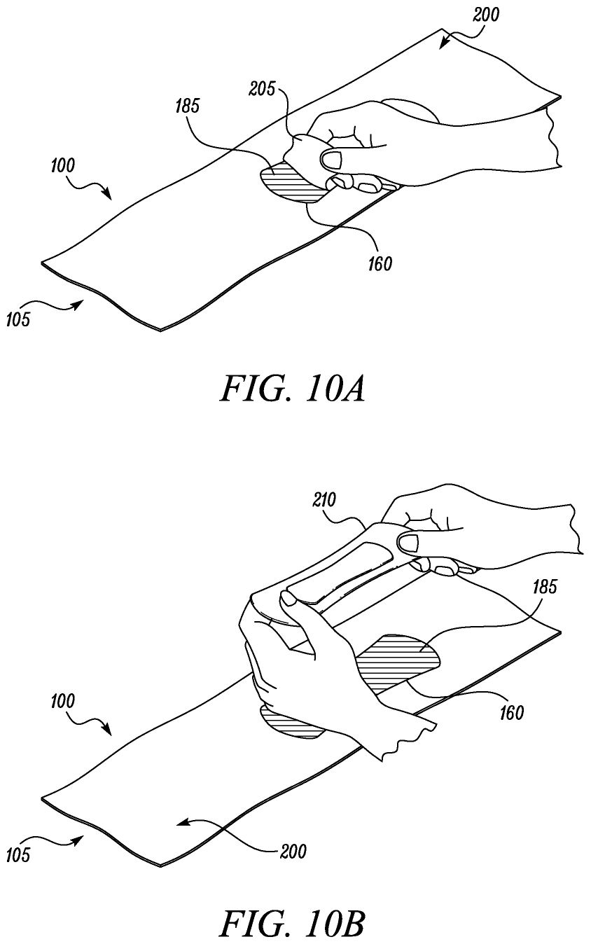

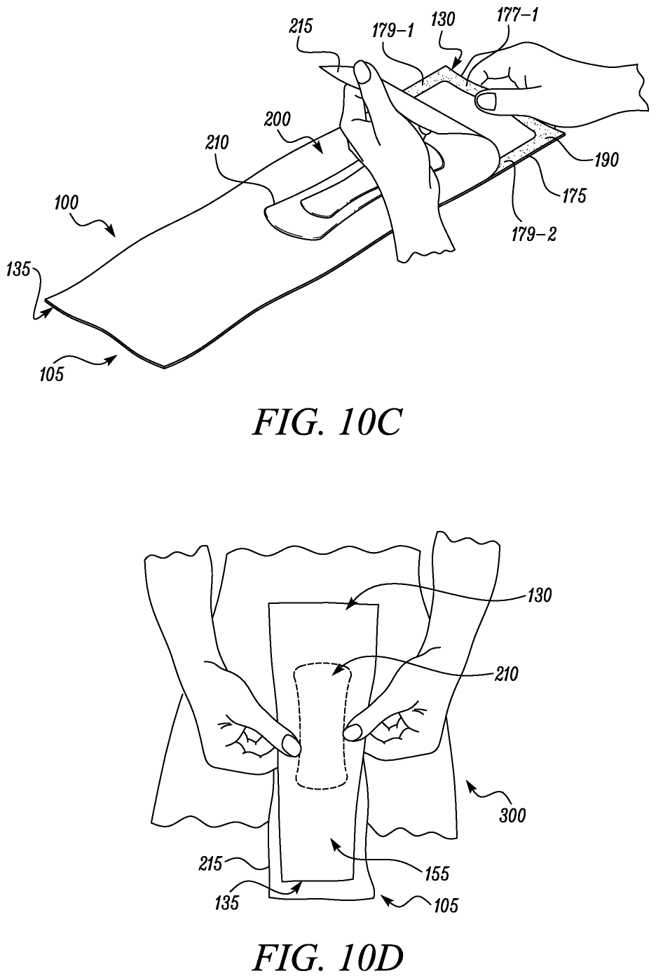

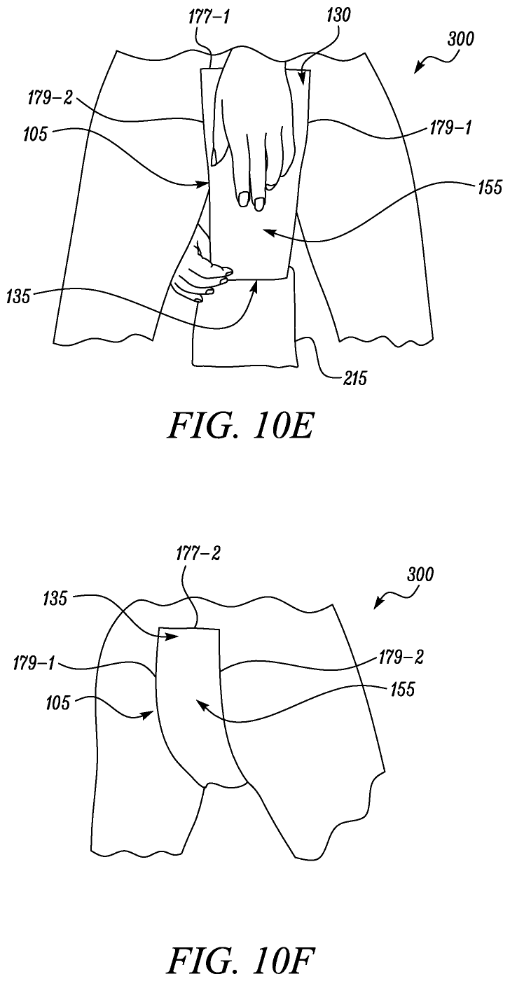

FIG. 9 illustrates an exemplary method 900 for securing the auxiliary band 100, 100′, 100″, 100′″ onto the skin surrounding the vulva region of a wearer 300, in accordance with some embodiments. To use the auxiliary band 100, 100′, 100″, 100′″ for securing the absorbent pad 210 onto the vulva region of the wearer 300, at 902, the wearer 300 peels the first cover portion 205 of the protective cover 200 to expose the first adhesive 185, 185′ disposed on the pad section 160 of the elongated body 105, as shown in FIG. 10A. In some embodiments, the visible indicators disposed on the pad section 160 of the elongated body 105 are also exposed, when the wearer 300 peels the first cover portion 205 of the protective cover 200.

After the first cover portion 205 is removed from the elongated body 105, at 904, the absorbent pad 210 is removably secured to the pad section 160 of the elongated body 105 as shown in FIG. 10B. The first adhesive 185, 185′ disposed on the pad section 160 securely attaches the absorbent pad 210 to the pad section 160. The visible indicators provide placement aid to suitably place the absorbent pad 210 on the exposed first adhesive 185, 185′ on the pad section 160.

In an alternate embodiment (not illustrated) the absorbent pad 210 is permanently built into the pad section 160 of the elongated body 105 at the time of manufacture. In such alternate embodiment, the method 900 begins as described hereinbelow with FIG. 10C.

After the absorbent pad 210 is attached to the pad section 160, at 906, the wearer 300 starts peeling the second cover portion 215 of the protective cover 200 from the front end 130 to expose the second adhesive 190 disposed on the front end 130 of the peripheral portion 175 as shown in FIG. 10C. After the second cover portion 215 of the protective cover 200 is removed from the front end 130, at 908, the wearer 300 attaches the exposed second adhesive 190 on the front end 130, including, but not limited to the lateral edge 177-1 of the inner surface 150 to her mons pubis region, as shown in FIG. 10D.

Further, at 910, the wearer 300 then continues to remove the second cover portion 215 of the protective cover 200 progressively from the front end 130 towards the rear end 135 to expose the second adhesive 190 disposed on the rear end of the elongated body 105, as shown in FIG. 10E. In some embodiments, the second cover portion 215 when removed, also exposes the second adhesive 190 disposed on the longitudinal edges 179-1, 179-2 and the third adhesive 195 disposed on the intermediary portion 180 of the elongated body 105.

Further, at 912, the wearer 300 wears the auxiliary band 100, 100′, 100″, 100′″ by attaching the rear end 135, including, but not limited to the lateral edge 177-2 of the inner surface 150 to her tailbone region, as shown in FIG. 10F. The longitudinal edges 179-1, 179-2 of the inner surface 150 are also attached to the skin between the mons pubis region and the tailbone region, as shown in FIG. 10F. In some embodiments, when the third adhesive 195 is disposed on the intermediary portion 180 of the elongated body 105, the exposed third adhesive 195 is also attached on the area surrounding the vulva region of the wearer 300.

After the elongated body 105 is applied to the area surrounding the vulva region of the wearer 300, the wearer 300 may check for proper positioning of the elongated body 105 on the area surrounding the vulva region and if required, the wearer 300 may remove and replace the elongated body 105 on the area surrounding the vulva region for better alignment.

In some embodiments, when the first adhesive 185, 185′, the second adhesive 190, and the third adhesive 195 include the plurality of layer of adhesives, a top layer of the plurality of layer of adhesives is removed to expose another layer of adhesive lying underneath, after a period of usage. The other layer of adhesive may be exposed by peeling off the top layer of the adhesive. After the top layer of the adhesive is removed and the other layer of the adhesive is exposed, the other layer of adhesive may be used to re-secure the absorbent pad 210 on the pad section 160 and/or the auxiliary band 100, 100′, 100″, 100′″ to the skin of the wearer 300, as already discussed.

Broadly, embodiments provide an apparatus and method for securing an absorbent pad 210, such as menstrual pad, panty liner, incontinence absorbent article, and the like, onto a vulva region of the wearer 300. As stated above, the existing sanitary products, such as, the absorbent pads and tampons, are either unsuitable or uncomfortable for wearing during sports and/or underwater activities.

The embodiments described herein provide a non-invasive, easy to use, and waterproof auxiliary band 100, 100′, 100″, 100′″ that safely and tightly secures the absorbent pad 210 onto the vulva region of the wearer 300. More specifically, the embodiments provide the auxiliary band 100, 100′, 100″, 100′″ that is designed to receive the absorbent pad 210 on its inner surface 150 and securely attach itself, along with the absorbent pad 210, directly onto the skin surrounding the vulva region of the wearer 300. Since the front end 130 and the rear end 135 are attached to the skin of the wearer 300 from all sides, the absorbent pad 210 can be firmly kept in place and in proper contact with the vulva region of the wearer 300, thereby preventing any leakage of the bodily fluid, during sports or underwater activities.

Moreover, since the auxiliary band 100, 100′, 100″, 100′″ is made from waterproof material, the auxiliary band 100, 100′, 100″, 100′″ acts as a barrier between the water and the absorbent pad 210 during underwater activities and prevents the water from entering the absorbent pad 210 secured to the inner surface 150, while also preventing the bodily fluid accumulated in the absorbent pad 210 from leaking out of the auxiliary band 100, 100′, 100″, 100′″.

The auxiliary band 100, 100′, 100″, 100′″ may also be made from a flexible material to conform to the skin surrounding the vulva region of the wearer 300. In an exemplary embodiment, the flexible material may extend to the inner thighs of the wearer 300 to firmly support the absorbent pad 210 on the skin of the wearer 300. In some embodiments, the auxiliary band 100, 100′, 100″, 100′″ or specifically the area of the auxiliary band 100, 100′, 100″, 100′″ that extends along the inner thighs of the wearer 300 may be made from a transparent material so that the material blends with the color of the skin of the wearer 300. This allows the auxiliary band 100, 100′. 100″, 100′″ to be worn nearly under any clothing, such as a swimsuit, without worrying about anyone noticing the application of the auxiliary band 100, 100′, 100″, 100′″.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, front and rear, and the like may be used solely to distinguish one entity or action from another entity or action without necessarily requiring or implying any actual such relationship or order between such entities or actions. The terms “comprises.” “comprising,” “has”, “having,” “includes”, “including.” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element preceded by “comprises . . . a”, “has . . . a”. “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

We claim:

1. An auxiliary band for securing an absorbent pad onto a vulva region of a female, the auxiliary band comprising:
an elongated body including:
an inner surface adapted to face the vulva region, the inner surface defining:
a pad section adapted to receive the absorbent pad thereon, and
a peripheral portion extending along an outer periphery of the inner surface;
a first adhesive disposed on the pad section, the first adhesive being adapted to removably secure the absorbent pad to the pad section; and
a second adhesive disposed at least on the peripheral portion, the second adhesive being adapted to removably attach the auxiliary band to the skin surrounding the vulva region.

2. The auxiliary band of claim 1, wherein the inner surface further defines an intermediary portion extending between the pad section and the peripheral portion, and wherein the auxiliary band comprises a third adhesive disposed on the intermediary portion, the third adhesive being adapted to removably attach the intermediary portion of the auxiliary band to the skin surrounding the vulva region.

3. The auxiliary band of claim 1, wherein each of the first adhesive and the second adhesive include a reusable adhesive being adapted to:
provide re-attachable adhesion for removably securing the absorbent pad to the pad section; and
provide re-attachable adhesion for removably securing the auxiliary band to the skin surrounding the vulva region of the female, respectively.

4. The auxiliary band of claim 1, wherein each of the first adhesive and the second adhesive are made of a same adhesive material.

5. The auxiliary band of claim 1, wherein each of the first adhesive and the second adhesive are made of different adhesive material.

6. The auxiliary band of claim 1 further comprising a protective cover for covering the first adhesive and the second adhesive, the protective cover being adapted to be removed to expose the first adhesive and the second adhesive for use.

7. The auxiliary band of claim 6, wherein the protective cover includes a first cover portion being adapted to be removed to expose the first adhesive and a second cover portion being adapted to be removed to expose the second adhesive.

8. The auxiliary band of claim 1, wherein the elongated body is made of silicone.

9. The auxiliary band of claim 1, wherein the second adhesive is made of a body adhesive material.

10. The auxiliary band of claim 1, wherein the elongated body includes a longitudinal axis defining a front end and a rear end of the auxiliary band, the front end being adapted to be removably attached to a mons pubis region and the rear end being adapted to be removably attached to a tailbone region of the female.

11. The auxiliary band of claim 1, wherein a length of the pad section is less than a length of the elongated body and wherein a width of the pad section is less than or equal to a width of the elongated body.

12. The auxiliary band of claim 1, wherein one or more of the first adhesive and the second adhesive include a plurality of layers of adhesive material, and wherein each layer of adhesive is adapted to be removed, after use, to expose another layer of adhesive lying underneath.

13. The auxiliary band of claim 1, wherein one or more of the first adhesive and the second adhesive are removably attached to the inner surface of the elongated body of the auxiliary band.

14. The auxiliary band of claim 1, wherein the absorbent pad is one of a menstrual pad, a panty liner, or an incontinence absorbent article.

15. A method for securing an absorbent pad onto a vulva region of a female, the method comprising:
providing an auxiliary band including:
an elongated body having an inner surface for facing the vulva region, the inner surface defining a pad section for receiving the absorbent pad thereon and a peripheral portion extending along an outer periphery of the inner surface;
a first adhesive disposed on the pad section, the first adhesive being adapted to removably secure the absorbent pad to the pad section;
a second adhesive disposed at least on the peripheral portion, the second adhesive being adapted to removably attach the auxiliary band to the skin surrounding the vulva region; and
a protective cover covering the first adhesive and the second adhesive;
peeling the protective cover to expose the first adhesive at the pad section and the second adhesive at the peripheral portion;
attaching the absorbent pad to the exposed first adhesive; and
attaching the exposed second adhesive onto the skin surrounding the vulva region.

16. The method of claim 15, wherein the protective cover includes a first cover portion and a second cover portion, and wherein the peeling the protective cover further comprising:
peeling the first cover portion to expose the first adhesive; and
peeling the second cover portion, after attaching the absorbent pad, to expose the second adhesive.

17. The method of claim 15, wherein the elongated body includes a longitudinal axis defining a front end and a rear end of the auxiliary band, and wherein attaching the exposed second adhesive onto the skin further comprising:
attaching the front end to a mons pubis region; and
attaching the rear end to a tailbone region of the female.

18. The method of claim 15, wherein the first adhesive includes a plurality of layers of adhesive material, and wherein the attaching the absorbent pad further comprising:
removing a top layer of first adhesive to expose another layer of adhesive lying underneath.

19. The method of claim 15, wherein the second adhesive includes a plurality of layers of adhesive material, and wherein the attaching the second adhesive onto the skin further comprising:
removing a top layer of second adhesive to expose another layer of adhesive lying underneath.

20. The method of claim 15, wherein the elongated body is made of silicone and wherein the second adhesive is made of a body adhesive material.
